(19) United States
(12) Patent Application Publication
(10) Pub. No.: US 2024/0285215 A1
(43) Pub. Date: Aug. 29, 2024

(54) DEVELOPMENT OF A BEAT-TO-BEAT FETAL ELECTROCARDIOGRAM

(71) Applicant: CHILDREN'S NATIONAL MEDICAL CENTER, Washington, DC (US)

(72) Inventors: Anita KRISHNAN, Washington, DC (US); Rathinaswamy B. GOVINDAN, Washington, DC (US); Pooneh ROSHANITABRIZI, Washington, DC (US)

(73) Assignee: CHILDREN'S NATIONAL MEDICAL CENTER, Washington, DC (US)

(21) Appl. No.: 18/686,890

(22) PCT Filed: Oct. 14, 2022
(86) PCT No.: PCT/US2022/046740

§ 371 Date: Feb. 27, 2024

Related U.S. Application Data
(60) Provisional application No. 63/256,436, filed on Oct. 15, 2021.

(51) Int. Cl. A61B 5/344; A61B 5/00; A61B 5/346

(52) U.S. Cl. CPC A61B 5/344; A61B 5/346; A61B 5/7221

(57) ABSTRACT

A method for extracting a fetal electrocardiogram (ECG). comprising determining, via processing circuitry, a coherence between a maternal ECG and an abdominal ECG, attenuating, via the processing circuitry, the maternal ECG independent of the coherence, and extracting, via the processing circuitry, the fetal ECG from the abdominal ECG based on the coherence and the attenuated maternal ECG.

### CROSS-REFERENCE TO RELATED APPLICATIONS

The present application claims priority to U.S. Provisional Application No. 63/256,436, filed Oct. 15, 2021, which is incorporated herein by reference in its entirety for all purposes.

### BACKGROUND

### Field of the Disclosure

The present disclosure relates to automatic extraction of a fetal electrocardiogram.

### Description of the Related Art

Fetal arrhythmia and other cardiac abnormalities can lead to severe health issues for both the fetus and the parent. Monitoring fetal cardiac activity is difficult due to lack of direct access to the fetus and movement of both the parent carrying the fetus and the fetus itself. Extracting a fetal electrocardiogram (ECG) from external ECG measurements taken outside of the womb can also be difficult due to overlap and interference from the maternal ECG. There is no clinical standard for acquiring and isolating a fetal ECG signal for analysis of fetal cardiac activity. Accuracy, speed, and sensitivity are exemplary factors that affect ease of use and clinical adoption of fetal ECG extraction methods.

The foregoing “Background” description is for the purpose of generally presenting the context of the disclosure. Work of the inventors, to the extent it is described in this background section, as well as aspects of the description which may not otherwise qualify as prior art at the time of filing, are neither expressly nor impliedly admitted as prior art against the present invention.

### SUMMARY

In one embodiment, the present disclosure is related to a method for extracting a fetal electrocardiogram (ECG), comprising determining, via the processing circuitry, a coherence between a maternal ECG and an abdominal ECG; attenuating, via the processing circuitry, the maternal ECG independent of the coherence; and extracting, via the processing circuitry, the fetal ECG from the abdominal ECG based on the coherence and the attenuated maternal ECG.

In one embodiment, the present disclosure is related to a device comprising processing circuitry configured to determine a coherence between a maternal ECG and an abdominal ECG, attenuate the maternal ECG independent of the coherence, and extract a fetal ECG from the abdominal ECG based on the coherence and the attenuated maternal ECG.

In one embodiment, the present disclosure is related to a non-transitory computer-readable storage medium for storing computer-readable instructions that, when executed by a computer, cause the computer to perform a method, the method comprising determining a coherence between a maternal ECG and an abdominal ECG, attenuating the maternal ECG independent of the coherence, and extracting the fetal ECG from the abdominal ECG based on the coherence and the attenuated maternal ECG.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is a method for extraction of a fetal ECG, according to an exemplary embodiment of the present disclosure;

FIG. 2 is an illustration of an ECG lead montage, according to an exemplary embodiment of the present disclosure;

FIG. 3 is an illustration of an extracted and enhanced fetal ECG, according to an exemplary embodiment of the present disclosure;

FIG. 4A is an illustration of an abdominal ECG, according to an exemplary embodiment of the present disclosure;

FIG. 4B is an illustration of a maternal ECG, according to an exemplary embodiment of the present disclosure;

FIG. 4C is an illustration of an extracted fetal ECG, according to an exemplary embodiment of the present disclosure;

FIG. 4D is an illustration of an enhanced fetal ECG, according to an exemplary embodiment of the present disclosure;

FIG. 5A is an illustration of a public domain abdominal ECG, according to an exemplary embodiment of the present disclosure;

FIG. 5B is an illustration of a public domain abdominal ECG (channel 4), according to an exemplary embodiment of the present disclosure;

FIG. 5C is an illustration of an extracted fetal ECG, according to an exemplary embodiment of the present disclosure;

FIG. 5D is an illustration of an enhanced fetal ECG, according to an exemplary embodiment of the present disclosure;

FIG. 6A is an illustration of a synthetic abdominal ECG, according to an exemplary embodiment of the present disclosure;

FIG. 6B is an illustration of a synthetic maternal ECG, according to an exemplary embodiment of the present disclosure;

FIG. 6C is an illustration of a synthetic fetal ECG, according to an exemplary embodiment of the present disclosure;

FIG. 6D is an illustration of an extracted fetal ECG, according to an exemplary embodiment of the present disclosure;

FIG. 7 is a schematic of a user device for performing a method, according to an exemplary embodiment of the present disclosure;

FIG. 8 is a schematic of a hardware system for performing a method, according to an exemplary embodiment of the present disclosure; and

FIG. 9 is a schematic of a hardware configuration of a device for performing a method, according to an exemplary embodiment of the present disclosure.

### DETAILED DESCRIPTION

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). Reference throughout this document to “one embodiment”, “certain embodiments”, “an embodiment”, “an implementation”, “an example” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

In one embodiment, the present disclosure is directed to systems and methods for automatic detection and analysis of fetal cardiac activity. The analysis of fetal cardiac activity can include detection of abnormalities, including arrhythmias, that can lead to severe health issues or even death. It is estimated that 1-3% of pregnancies experience fetal arrhythmias, and it is suspected that fetal rhythm disorders cause at least 3-10% of unexplained stillbirths. Fetal arrhythmias and repolarization abnormalities can progress to hydrops fetalis or other conditions, which are associated with increased fetal mortality and preterm delivery. However, appropriate detection and treatment of arrhythmias can prevent conditions such as hydrops fetalis and lead to a favorable outcome for the fetus and the parent, with up to 96% survival.

Fetal cardiac monitoring is typically performed through various specialized techniques such as cardiotocography, magnetocardiography, and Doppler ultrasound. These methods are used to acquire fetal signals but are inconvenient and are not suitable for continuous, portable monitoring, which may be needed to regularly assess fetal cardiac rhythms throughout a pregnancy. In addition, methods such as magnetocardiography require a costly shielding environment. Electrocardiogram (ECG) acquisition is a common method for detecting arrhythmias in pediatric and adult patients due to its moderate cost and accessibility in healthcare facilities but is not routinely used for fetal monitoring. Fetal ECG technology relies on signal averaging and is primarily designed for heart rate monitoring in fetuses after 36 weeks of gestation, which is near the end of a typical gestation period. In addition, noninvasive fetal ECG acquisition is challenging due to lack of direct contact with the fetus and the overlap between a low-amplitude (weak) fetal ECG and a comparatively high-amplitude (strong) maternal ECG. Additional disturbances such as the presence of amniotic fluid, muscle activity, respiration, movement, changes in orientation or position, and background noise, including background noise in the womb, can also cause inaccurate readings that are difficult to assess.

Separation of fetal ECG from maternal ECG is an important area of interest for improving fetal ECG monitoring. Signal processing techniques for separation should be easy to implement, require single channel inputs, and be effective and accurate for small windows of data in order to translate easily to clinical use. Certain signal processing techniques can require multiarray (multichannel) data in order to decompose a raw signal into independent components or can be highly dependent on consistent maternal ECG morphology, which may be difficult due to maternal and/or fetal movement and breathing during acquisition. These techniques are overly sensitive to changes and noise during ECG acquisition that can lead to significant baseline wandering, noise, and inaccuracies, especially in template detection and matching. As a result, none of these methods have been adapted as standard clinical tools. There is therefore a need for an effective and accurate method for separation of fetal ECG from maternal ECG and further processing to assess an isolated fetal ECG.

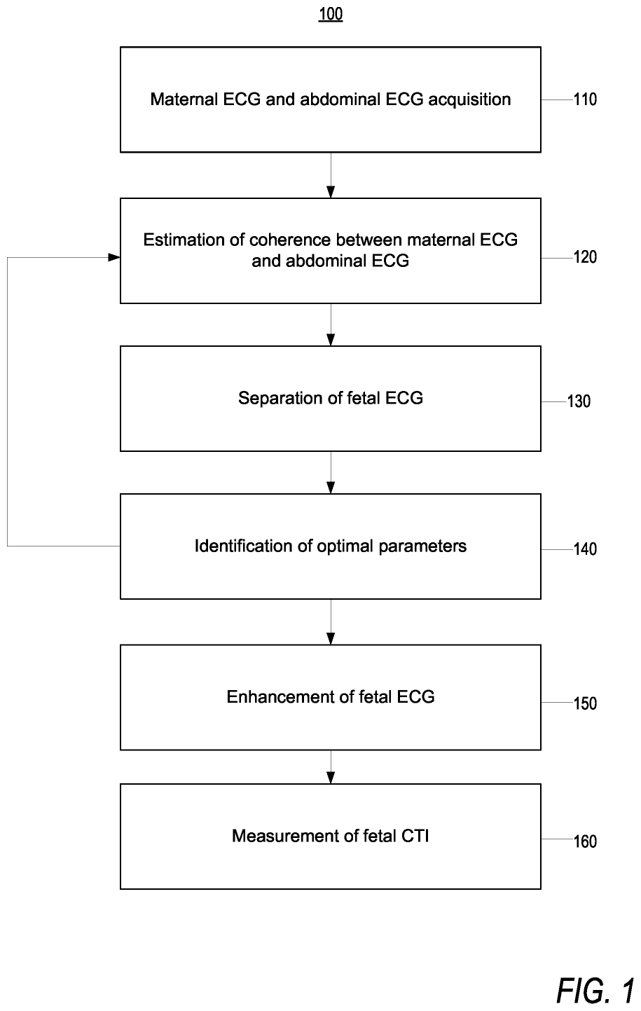

In one embodiment, the present disclosure is directed to a frequency-based approach for early fetal ECG analysis. FIG. 1 illustrates a method 100 for fetal ECG analysis, according to one embodiment of the present disclosure. In step 110, the maternal ECG and the abdominal ECG can be acquired. In step 120, the coherence between the maternal ECG and the abdominal ECG can be calculated based on the maternal and abdominal readings. According to one embodiment, the coherence can be determined in the frequency domain. In step 130, the fetal ECG can be separated or extracted from the maternal ECG based on the coherence between the maternal ECG and the abdominal ECG. In step 140, the optimal parameters of the fetal ECG can be determined and used to confirm the extracted fetal ECG. If the fetal ECG is not optimal, the process of estimating coherence in step 120 and separating the fetal ECG in step 130 can be repeated until the fetal ECG is an optimal signal. In step 150, the fetal ECG can be enhanced to improve signal quality. In one embodiment, the enhancement can be applied in the time domain. In step 160, cardiac time intervals (CTIs) can be determined for the fetal ECG. The CTIs can include, but are not limited to, PR, QRS, RR, QT, corrected QT (QTc) intervals. Each of these steps will be described in further detail herein.

In one embodiment, the methods of the present disclosure can include signal acquisition of a maternal ECG and an abdominal ECG using ECG electrodes (leads) and a data acquisition system. One or more channels of maternal ECG and one or more channels of abdominal ECG can be used to extract a fetal ECG. Each channel can include an ECG reading from a different position. The position and placement of the leads can affect the recorded ECG for each channel. According to one example, 5-7 channels of maternal ECG and 8-16 channels of abdominal ECG can be recorded. The abdominal ECG includes a mix of the maternal ECG and the fetal ECG. The trace of the maternal ECG found in the abdominal ECG can vary from the independently recorded maternal ECG. For example, the maternal ECG can be attenuated in the abdominal ECG due to the distance from the heart.

In one embodiment, the position of the ECG electrodes can be independent of the fetal position. Thus, the fetal position does not have to be determined via imaging or other methods before the ECG is acquired. The methods of the present disclosure can be used for various lead positions and vector montages. FIG. 2 is an illustration of a lead montage for maternal and abdominal ECG acquisition, according to one embodiment of the present disclosure. The montage in FIG. 2 is a non-limiting example. The methods described herein can be applied to ECG data independently of the montage that is used. In one embodiment, the electrodes can be densely arranged to avoid loss in fetal ECG due to fetal motion. In one embodiment, the ECG can be acquired for approximately five minutes. Longer and shorter periods of acquisition can also be compatible with the methods of the present disclosure. Various sampling rates can be used. For example, 250 Hz, 500 Hz, 750 Hz, 1 kHz, 2 kHz, etc. The performance of the disclosed methods is not dependent on ECG sampling frequency. According to one embodiment, additional electrodes and/or readings can be acquired. For example, a fetal scalp electrode can be used to acquire additional data to be used as references for peak or wave identification. According to one embodiment, the ECGs can be acquired at any point throughout the gestational period from approximately 21 weeks to approximately 40 weeks. Earlier and later acquisitions can also be compatible with the present methods. The methods of the present disclosure can be effective for a wide range of heart rates, sampling frequencies, cardiac waveforms and morphologies, and noise levels.

An abdominal ECG from an abdominal ECG channel and a maternal ECG from a maternal ECG signal can be selected for analysis. In one embodiment, the abdominal ECG and maternal ECG can be pre-processed to remove noise and baseline wandering. The pre-processing can include, for example, filtering, decomposition, and/or morphology analysis. According to one example, the pre-processing can include filtering the abdominal ECG and the maternal ECG signals, for example, using a high-pass, fourth-order Butterworth filter with a 0.5 Hz cutoff frequency. The filter can be applied to forward and reverse directions of the signals. The abdominal ECG can be regarded as a reference signal, and the maternal ECG can be regarded as a source signal.

Coherence between the maternal ECG and the abdominal ECG can be determined in order to quantify the presence of the maternal ECG in the abdominal ECG. Coherence analysis can refer to the estimation of a relationship or concordance between two systems. The coherence can be quantified using a coherence function. In one embodiment, the coherence function can estimate the extent to which a first function or signal can be used to predict a second function or signal.

Thus, the coherence between the maternal ECG and the abdominal ECG can indicate the effect of the maternal ECG on the abdominal ECG. Values and waveforms in the abdominal ECG that are not caused by or associated with the maternal ECG can then be associated with the fetal ECG. Values of coherence can fall between 0 and 1, inclusively. In one embodiment, coherence analysis can further include identification of coherent components present in the maternal ECG and the abdominal ECG.

According to one embodiment, the coherence between the maternal ECG and the abdominal ECG can be determined using the maternal ECG and the abdominal ECG signals in the frequency domain. In one embodiment, the ECG signals can be transformed to the frequency domain using a Fourier transform. In one embodiment, the signals can be divided into inspection windows for analysis. The inspection windows can further be divided into epochs. For example, an inspection window can be a 1-minute inspection window. The abdominal ECG signal and the maternal ECG signal in the 1-minute inspection window can further be split, for example, into twenty 3-second epochs, wherein 3 seconds is the Fourier transform length for an optimal spectral estimate. Each epoch is thus associated with an abdominal ECG signal and a maternal ECG signal, wherein the abdominal ECG signal and the maternal ECG signal are synchronized in the epoch. For each signal, a mean value can be calculated over the inspection window or over the length of the full signal. The mean value can then be subtracted from the data in each epoch. In one embodiment, the mean can be subtracted to remove a baseline from each epoch. The signals can then be transformed to the frequency domain using the Fourier transform. Subsequent processing can then be performed in the frequency domain until the signals are transformed back to the time domain.

According to one embodiment, the coherence between the abdominal ECG and the maternal ECG can be calculated for each epoch using a cross-spectrum between each periodogram associated with the epoch. In one embodiment, the coherence can be estimated using the Welch periodogram approach. The periodogram is a function of the Fourier transform used to identify dominant frequencies and estimate the spectral density of each epoch. In one embodiment, the periodogram for abdominal ECG S_{aECG}^{j} and the periodogram for maternal ECG S_{mECG}^{j} for the jth epoch can be calculated as in Equations 1 and 2:

$${S}_{aECG}^{j} ( ω ) = {\vert {F}_{aECG}^{j} ( ω ) \vert}^{2} , \qquad ( 1 )$$

$${S}_{mECG}^{j} ( ω ) = {\vert {F}_{mECG}^{j} ( ω ) \vert}^{2} , \qquad ( 2 )$$

wherein F_{aECG}^{j} denotes the Fourier transform of abdominal ECG and F_{mECG}^{j} denotes the Fourier transform of maternal ECG for the jth epoch and ω is a frequency in Hz. The magnitude of the Fourier transform is taken with the | | operator. The cross-spectrum S_{aECG,mECG}^{j} between the abdominal ECG periodogram S_{aECG}^{j} and the maternal ECG periodogram S_{mECG}^{j} can be calculated as in Equation 3:

$${S}_{aECG , mECG}^{j} ( ω ) = {F}_{aECG}^{j} ( ω ) \times {F}_{mECG}^{j} ( ω ) , \qquad ( 3 )$$

wherein [symbol] represents the complex conjugate operator. The coherence between the abdominal ECG and the maternal ECG can then be calculated using spectral coherence. According to one embodiment, spectral coherence can be defined as in Equation 4:

$${Coh}_{aECG , mECG} ( ω ) = \frac{{\vert {\sum }_{j = 1}^{N} {S}_{aECG , mECG}^{j} ( ω ) \vert}^{2}}{( {\sum }_{j = 1}^{N} {S}_{aECG}^{j} ( ω ) \times {\sum }_{j = 1}^{N} {S}_{mECG}^{j} ( ω ) )} . \qquad ( 4 )$$

The coherence value Coh_{aECG,mECG}(ω) can be within the range [0, 1], wherein 0 indicates asynchrony between the two signals and 1 indicates complete synchrony between the two signals. In one example, a coherence value of 1 indicates that the equations can be described as a linear system with a single input function (e.g., maternal ECG) and a single output function (e.g., abdominal ECG).

In one embodiment, the confidence level of the coherence can be calculated using the equation 1−(1−α)^{1/(N-1}), wherein Nis a number of epochs involved in the spectral estimation and a is a significance level. In one embodiment, α can be 0.99 according to some references. According to one embodiment, the fetal ECG can be extracted only if the coherence is determined to be significant based on the confidence level. In one example, the combination of maternal ECG and abdominal ECG channels used can be a combination that yields a desired coherence confidence level.

In one embodiment, the fetal ECG signal can be extracted using an impulse-response transfer function. The maternal ECG can be identified and distinguished in the abdominal ECG based on the coherence between the abdominal ECG and the maternal ECG. The epoch division and analysis of signals ensures that the coherence between the abdominal ECG and the maternal ECG is based on the same moment in time for both ECG signals. Thus, variations in signals over time due to movement, noise, etc. are accounted for because the two signals are synchronized in each epoch. In one embodiment, the impulse-response transfer function H_{aECG,mECG}(ω) can be defined based on the cross-spectrum between the two periodograms, as in Equation 5:

$${H}_{aECG , mECG} ( ω ) = \frac{{\sum }_{j = 1}^{N} {S}_{aECG , mECG}^{j} ( ω )}{{\sum }_{j = 1}^{N} {S}_{mECG}^{j} ( ω )} . \qquad ( 5 )$$

The impulse-response transfer function can be calculated using a summation of values across all epochs. The impulse-response transfer function can be used to attenuate the maternal ECG and remove the coherent components between the maternal ECG and the abdominal ECG for each epoch. The maternal ECG is attenuated to correct for differences between the original maternal ECG signal and the maternal ECG signal present in the abdominal ECG. The differences can be, for example, due to the distance between the abdominal ECG leads and the maternal heart. The subtraction of the attenuated maternal ECG signal results in the fetal ECG F_{fECG}^{j}(ω) being extracted as the remaining signal as in Equation 6:

$${F}_{fECG}^{j} ( ω ) = {F}_{aECG}^{j} ( ω ) - ( {H}_{aECG , mECG}^{} ( ω ) ) \times ( {F}_{mECG}^{j} ( ω ) ) , \qquad ( 6 )$$

The attenuation of the maternal ECG and the extraction of the fetal ECG can be performed for each epoch j=1, j=2, . . . j=N.

In one example, the fetal ECG signal can be extracted from the abdominal ECG signal using graphical analysis of the abdominal ECG signal and/or the maternal ECG signal. A machine learning model can identify maternal ECG signal features in an abdominal ECG signal. The identified maternal ECG signal can be used for coherence estimation as has been described herein. According to one embodiment, the machine learning can include a deep learning model or an image analysis model. The machine learning model can be trained to identify and extract a maternal ECG signal from an abdominal ECG signal.

According to one embodiment, the fetal ECG F_{fECG}^{j}(ω) can be converted back to the time domain for optimal parameter identification and enhancement. In one embodiment, optimal parameters for the extraction of the fetal ECG F_{fECG}^{j}(ω) can be determined using a loss function.

A loss function can estimate a number of missed and extra beats in an ECG signal. The loss function should be minimized for a high-quality ECG signal. In one embodiment, a lower and upper bound for fetal heart rate can be used to determine a loss function. Low-amplitude signals that are not detected as fetal heartbeats in the abdominal ECG will result in a false fetal heart rate that is lower than the lower bound. Noisy signal can result in false detection of fetal heartbeats and a false fetal heart rate that is higher than the upper bound. The false detection of fetal heartbeats can include detection of maternal ECG signal in the fetal ECG signal, which can result in inaccurate extraction of the fetal ECG signal. According to one example, the fetal heart rate can be estimated to be between 105 to 190 bpm. The fetal heart rate estimation can be based on empirical studies and known fetal cardiac data.

In one embodiment, the loss function (l) can be determined to estimate the number of missed and extra beats. As an example, the loss function and the estimated missed and extra beats can be determined according to Equations 7-9:

$$l = missed + extra , \qquad ( 7 )$$

$$missed = ( \sum \frac{RRi}{median RR interval} ) - M , \qquad ( 8 )$$

$$extra = E - ( \sum \frac{RRe}{median RR interval} ) , \qquad ( 9 )$$

In one embodiment, RRi can indicate RR intervals longer than 0.5714 seconds and RRe can indicate RR intervals shorter than 0.3158 seconds, which are the respective periods of the lower and upper bounds for fetal heart rate as described above. Similarly, M can represent the total number of intervals exceeding 0.5714 seconds and E can represent the total number of intervals shorter than 0.3158 seconds. Additional or alternate values for RRi, RRe, M, and E can be used based on fetal heart rate bounds. Equations 8-9 have been developed to estimate a number of missed beats and a number of extra beats in a fetal ECG signal based on the number of identified RR intervals outside of the estimated fetal heart rate interval. In one embodiment, the missed beats and extra beats can be estimated using methods described by Wilson in Integrated Approach for Fetal QRS Detection, published September 2008 in IEEE Trans Biomed Eng. The RR interval can be the time elapsed between consecutive R waves on a fetal ECG. In one embodiment, the Pan-Tompkins method can be used to identify R waves and to calculate fetal heart rate. According to some embodiments, the fetal heart rate can be confirmed using an additional signal, such as a reading from a scalp electrode.

The loss function (l) can be calculated for each fetal ECG extracted from each combination of channels for maternal ECG acquisition and abdominal ECG acquisition. According to one embodiment, the optimal channel combination can be the combination that yields the minimum loss function. The optimal parameters for the system can include the optimal channel combination based on the minimum loss function. In one embodiment, the estimation of coherence and the separation of the fetal ECG can be repeated until the optimal parameters have been determined. For example, the coherence between maternal ECG and abdominal ECG and the separation of the fetal ECG can be performed for each combination of ECG channels. The loss function for each fetal ECG can be calculated. The optimal fetal ECG selected for further enhancement and analysis can be the fetal ECG associated with the minimum loss function.

According to one embodiment, machine learning can be used to determine the signal quality for fetal ECG extraction. For example, machine learning can be applied to identify the RR intervals or other cardiac time points and intervals based on the fetal ECG signal. The machine learning can include a deep learning model or an image analysis model. For example, a low-amplitude waveform in the fetal ECG signal can be an indication of a true, missed heartbeat. The machine learning model as described herein can be applied to identify the waveform as a missed heartbeat. The use of a machine learning model that has been trained with fetal cardiac datasets can prevent any errors or inaccuracies arising from thresholding and estimating cardiac signals. The machine learning model can be used to assess the fetal ECG signal for various quality metrics.

As an example, in an ECG lead arrangement with 2 maternal ECG channels and 2 abdominal ECG channels, a fetal ECG can be extracted from maternal ECG channel 1 and abdominal ECG channel 1, maternal ECG channel 1 and abdominal ECG channel 2, maternal ECG channel 2 and abdominal ECG channel 1, maternal ECG channel 2 and abdominal ECG channel 2. Four fetal ECGs can be extracted a loss function can be calculated for each of the four fetal ECGs. The fetal ECG associated with a minimum loss function out of the four calculated loss functions can be used for further analysis.

The fetal ECG can be enhanced to reduce any remaining presence of maternal ECG complexes and to improve analysis accuracy. In one embodiment, enhancement can include averaging of the fetal ECG. According to one implementation, the cardiogram can be averaged in time windows. A time window can include, for example, 0.5 seconds of data before each R wave and 0.6 seconds of data following each R wave. Shorter and longer time windows are compatible with the present method. In one embodiment, the time window that is averaged can include QRS complexes from previous and subsequent cycles. Additional or alternative waveforms or values can be included in the time window before and after each R wave. In one embodiment, the values within a selected time window can be averaged into a single value or more than one value. In one embodiment, beats or R waves that yield a heart rate outside of an expected fetal heart rate range (e.g., 105-190 bpm) can be excluded from the enhancement in order to ensure that genuine beats are used.

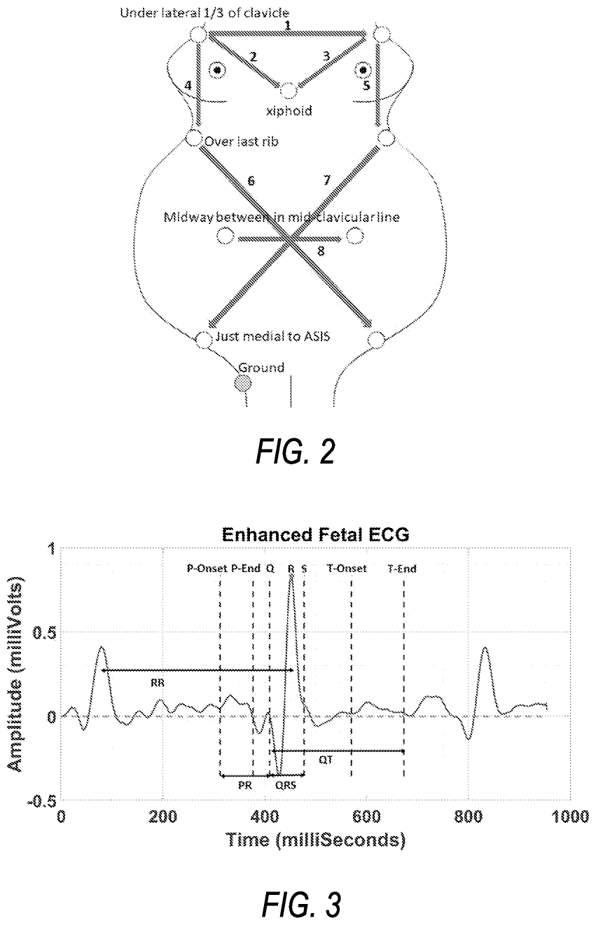

The fetal ECG can be analyzed after enhancement to determine fetal cardiac time points and fetal CTIs. Fetal cardiac time points can include, but are not limited to, a P-onset, a P-endpoint, a Q wave, an R wave, an S wave, a T-onset, and a T-endpoint. The fetal CTIs, such as the PR, QRS, RR, and QT intervals, can be determined based on the cardiac time points. In one embodiment, a corrected QT (QTc) interval can be calculated. For example, the QTc interval can be calculated based on the QT interval using the Bazett formula, QT_{c}=QT/\sqrt{RR}. In one embodiment, the cardiac time points and/or CTIs can be identified and calculated based on the fetal ECG manually, semiautomatically, or automatically, e.g., using machine learning (for example deep learning), computer vision, or image recognition. The fetal CTIs can then be used to identify arrhythmias or other cardiac abnormalities in the fetus. For example, a duration of a CTI can indicate cardiac abnormalities. FIG. 3 illustrates an example of an extracted and enhanced fetal ECG with cardiac time points and CTIs labeled.

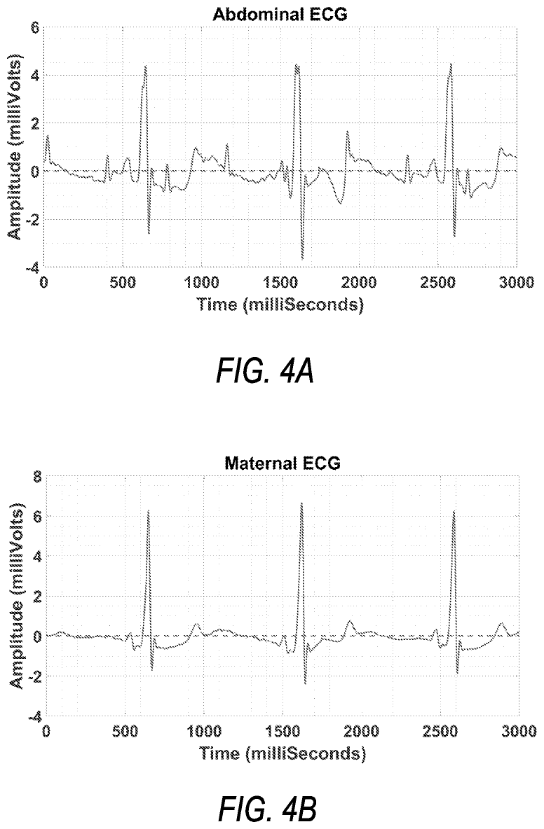

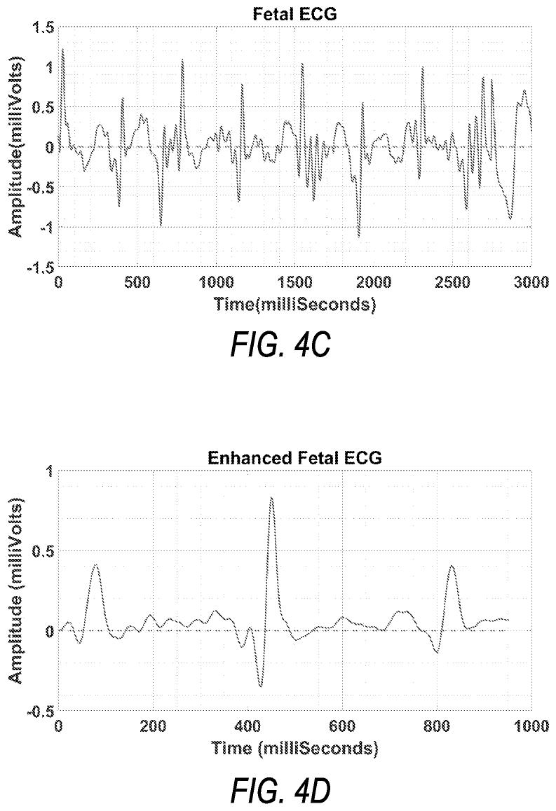

FIG. 4A through FIG. 4D illustrate an example of the signals acquired and extracted according to one embodiment of the present disclosure. FIG. 4A is an example of an acquired abdominal ECG. FIG. 4B is an example of a maternal ECG acquired along with the abdominal ECG. The abdominal ECG includes maternal ECG complexes as well as fetal ECG complexes. The coherence between the abdominal ECG of FIG. 4A and the maternal ECG of FIG. 4B can be determined as described in the present disclosure. The maternal ECG can then be attenuated and removed from the abdominal ECG to yield the fetal ECG, as shown in FIG. 4C. The amplitude of the fetal ECG is weaker than that of the maternal ECG, as can be seen in FIG. 4C. In some cases, the amplitude of the fetal ECG can be, for example, almost 10 times less than that of the maternal ECG. The difference in signal strength poses a challenge in properly attenuating the maternal ECG and extracting the fetal ECG. The use of the coherence analysis can result in accurate separation of the fetal ECG. The fetal ECG can then be enhanced to yield the signal of FIG. 4D. The enhanced signal includes distinctive waves that would be expected for an ECG and that can be used for CTI analysis. It can be seen from FIG. 4D that the maternal ECG complexes have been successfully removed from the fetal ECG through the coherence analysis and enhancement.

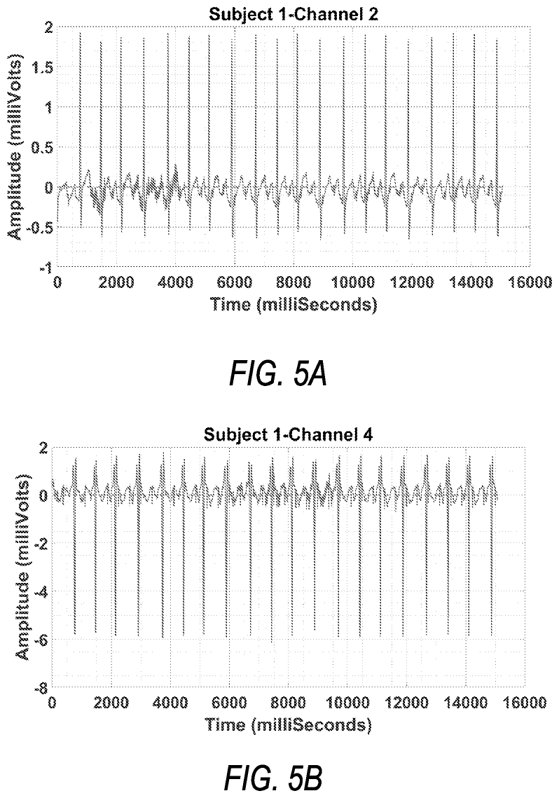

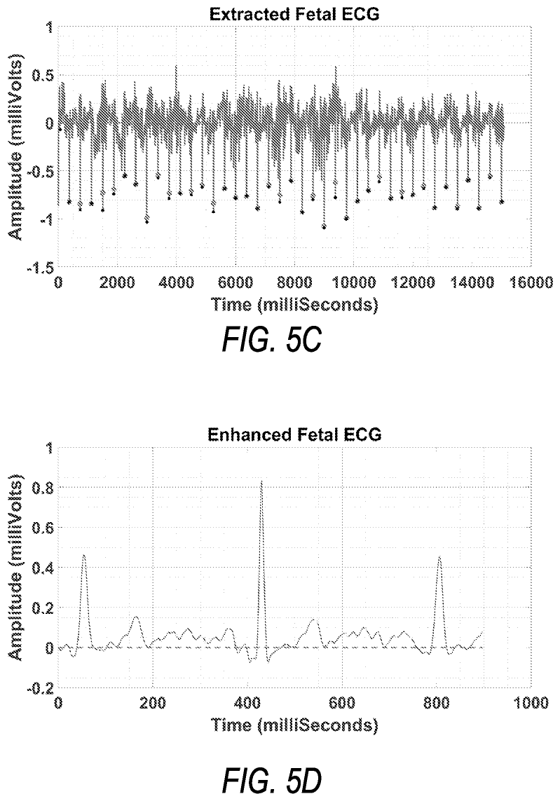

FIG. 5A through FIG. 5D illustrate an example of the signals acquired and extracted according to one embodiment of the present disclosure. FIG. 5A is an example of an acquired abdominal ECG. FIG. 5B is an example of an acquired maternal ECG. FIG. 5C is an example of an extracted fetal ECG, using the coherence analysis of the present disclosure. The extracted fetal ECG is labeled with the R peaks of the abdominal ECG as points along the fetal ECG. FIG. 5D is an example of an enhanced and reversed fetal ECG using the enhancements described herein. The enhanced fetal ECG is cleaner and the typical ECG waveforms can be clearly observed at low amplitudes in the fetal ECG.

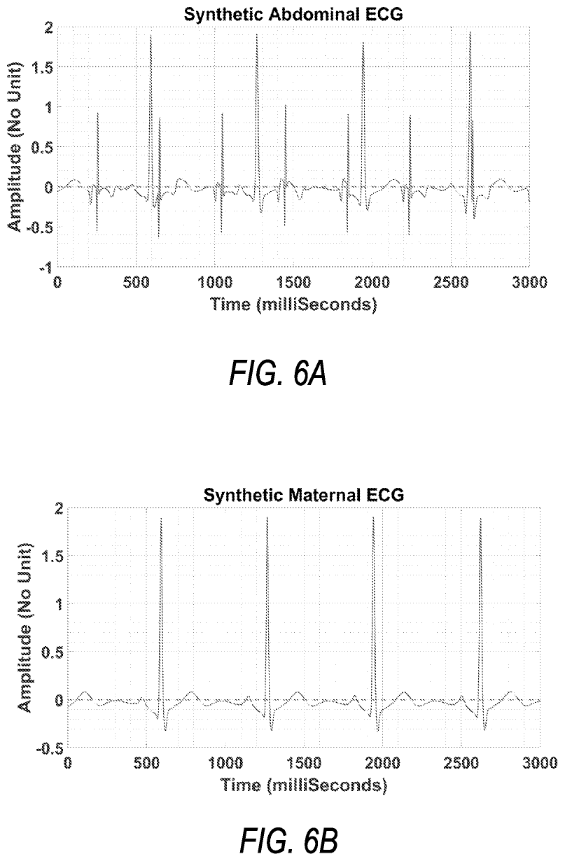

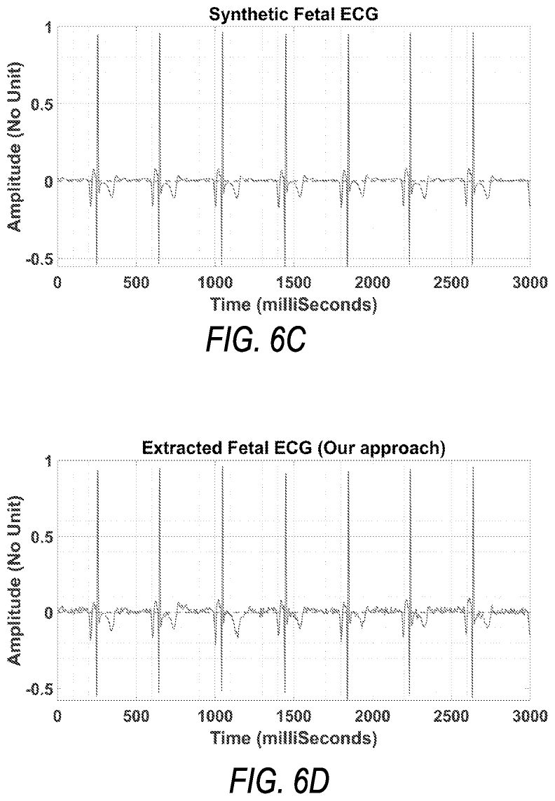

ECG readings can be assessed in a variety of methods, including visual inspection of the spectrum, CTI confirmation and agreement, and grading rubrics. In one implementation, the methods of the present disclosure yielded cleaner and more accurate fetal ECG signals than other methods. FIG. 6A through FIG. 6D illustrate an example of extracted fetal ECG using a synthetic ECG dataset. A synthetic fetal ECG as in FIG. 6C was combined with a synthetic maternal ECG to yield a synthetic abdominal ECG for analysis. The synthetic abdominal ECG is shown in FIG. 6A and the synthetic maternal ECG is shown in FIG. 6B. An embodiment of the coherence analysis method of the present disclosure was applied to extract the fetal ECG of FIG. 6D. The extracted fetal ECGs can be compared to the synthetic fetal ECG of FIG. 6C used to generate the synthetic abdominal ECG. Table 1 indicates a comparison of the methods of the present disclosure with blind source separation methods (fast independent component analysis (fastICA), principal component analysis (PCA)), template matching, and adaptive filtering (least means squared (LMS)). The extracted fetal ECG of FIG. 6D was correlated with the original synthetic fetal ECG of FIG. 6C. The fetal ECG extracted using coherence as described in the present disclosure had a higher correlation coefficient with the synthetic fetal ECG than the other methods, as shown in Table 1. The high correlation indicates that the extraction methods of the present disclosure preserve more features of the fetal ECG than other methods.

TABLE 1

| | | | | Template | |
|---|---|---|---|---|---|
| | Coherence | fastICA | PCA | matching | LMS |
| Correlation | 98.97% | 89.63% | 77.61% | 96.36% | 87.71% |
| coefficient | | | | | |

In one embodiment, the methods of the present disclosure can be executed at the machine level, e.g., in a machine-level language such as C or C++. Using a machine-level language can optimize the processing time for extracting the fetal ECG in a clinical setting. In some embodiments, the methods of the present disclosure can be performed in real time. The fetal ECG can be extracted and analyzed using noninvasive ECG acquisition, according to the present disclosure. In addition, the methods for extraction of the fetal ECG as described in the present disclosure have been shown to yield high-quality, reproducible ECGs for clinical assessment. The implementation of a fully automated and accurate method for fetal ECG extraction, as described herein, can improve assessment of fetal cardiac conditions in a clinical setting without requiring specialized equipment.

Embodiments of the subject matter and the functional operations described in this specification can be implemented by digital electronic circuitry, in tangibly embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory program carrier for execution by, or to control the operation of data processing apparatus. The computer storage medium can be a machine-readable storage device, a machine-readable storage substrate, a random or serial access memory device, or a combination of one or more of them.

The term “data processing apparatus” refers to data processing hardware and may encompass all kinds of apparatus, devices, and machines for processing data, including by way of example a programmable processor, a computer, or multiple processors or computers. The apparatus can also be or further include special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, Subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA an ASIC.

Computers suitable for the execution of a computer program include, by way of example, general or special purpose microprocessors or both, or any other kind of central processing unit. Generally, a CPU will receive instructions and data from a read-only memory or a random access memory or both. Elements of a computer are a CPU for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more Such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients (user devices) and servers. A client and server are generally remote from each other and typically interact through a communication network. The relationship of client and server arises by virtue of computer programs running on the respective computers and having a client-server relationship to each other. In an embodiment, a server transmits data, e.g., an HTML page, to a user device, e.g., for purposes of displaying data to and receiving user input from a user interacting with the user device, which acts as a client. Data generated at the user device, e.g., a result of the user interaction, can be received from the user device at the server.

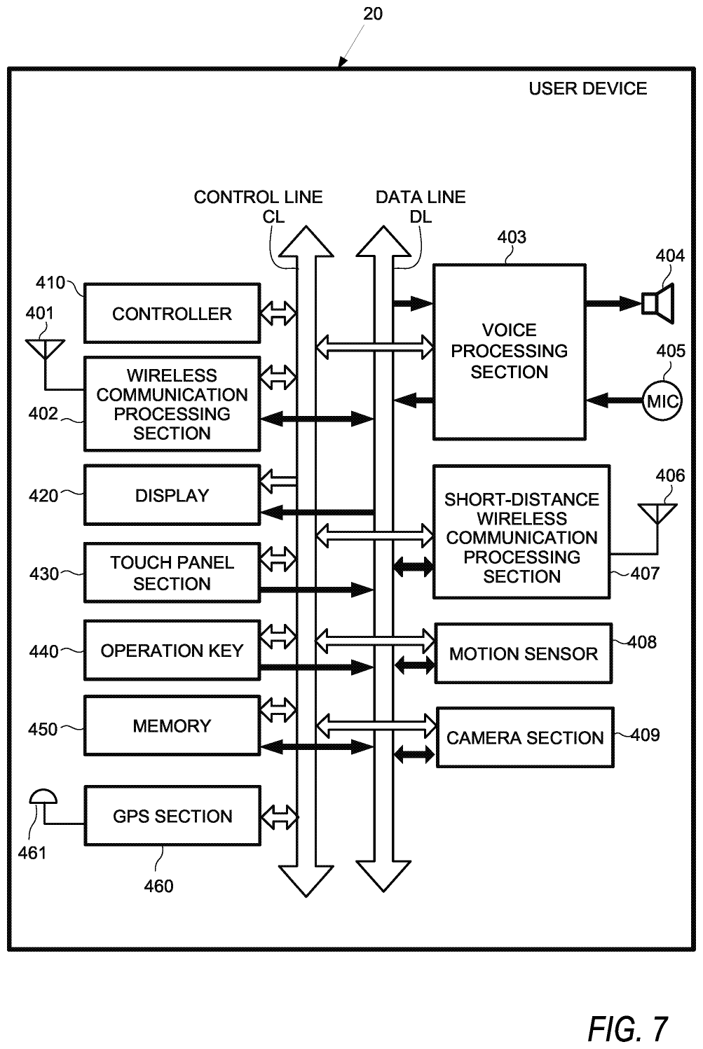

In an embodiment, the electronic user device 20 may be a smartphone. However, the skilled artisan will appreciate that the features described herein may be adapted to be implemented on other devices (e.g., a laptop, a tablet, a server, an e-reader, a camera, a navigation device, etc.). The exemplary user device 20 of FIG. 7 includes processing circuitry, as discussed above. The processing circuitry includes one or more of the elements discussed next with reference to FIG. 7. The electronic user device 20 may include other components not explicitly illustrated in FIG. 7 such as a CPU, GPU, frame buffer, etc. The electronic user device 20 includes a controller 410 and a wireless communication processor 402 connected to an antenna 401. A speaker 404 and a microphone 405 are connected to a voice processor 403.

The controller 410 may include one or more processors/processing circuitry (CPU, GPU, or other circuitry) and may control each element in the user device 20 to perform functions related to communication control, audio signal processing, graphics processing, control for the audio signal processing, still and moving image processing and control, and other kinds of signal processing. The controller 410 may perform these functions by executing instructions stored in a memory 450. Alternatively or in addition to the local storage of the memory 450, the functions may be executed using instructions stored on an external device accessed on a network or on a non-transitory computer readable medium.

The memory 450 includes but is not limited to Read Only Memory (ROM), Random Access Memory (RAM), or a memory array including a combination of volatile and non-volatile memory units. The memory 450 may be utilized as working memory by the controller 410 while executing the processes and algorithms of the present disclosure. Additionally, the memory 450 may be used for long-term storage, e.g., of image data and information related thereto.

The user device 20 includes a control line CL and data line DL as internal communication bus lines. Control data to/from the controller 410 may be transmitted through the control line CL. The data line DL may be used for transmission of voice data, displayed data, etc.

The antenna 401 transmits/receives electromagnetic wave signals between base stations for performing radio-based communication, such as the various forms of cellular telephone communication. The wireless communication processor 402 controls the communication performed between the user device 20 and other external devices via the antenna 401. For example, the wireless communication processor 402 may control communication between base stations for cellular phone communication.

The speaker 404 emits an audio signal corresponding to audio data supplied from the voice processor 403. The microphone 405 detects surrounding audio and converts the detected audio into an audio signal. The audio signal may then be output to the voice processor 403 for further processing. The voice processor 403 demodulates and/or decodes the audio data read from the memory 450 or audio data received by the wireless communication processor 402 and/or a short-distance wireless communication processor 407. Additionally, the voice processor 403 may decode audio signals obtained by the microphone 405.

The exemplary user device 20 may also include a display 420, a touch panel 430, an operation key 440, and a short-distance communication processor 407 connected to an antenna 406. The display 420 may be a Liquid Crystal Display (LCD), an organic electroluminescence display panel, or another display screen technology. In addition to displaying still and moving image data, the display 420 may display operational inputs, such as numbers or icons which may be used for control of the user device 20. The display 420 may additionally display a GUI for a user to control aspects of the user device 20 and/or other devices. Further, the display 420 may display characters and images received by the user device 20 and/or stored in the memory 450 or accessed from an external device on a network. For example, the user device 20 may access a network such as the Internet and display text and/or images transmitted from a Web server.

The touch panel 430 may include a physical touch panel display screen and a touch panel driver. The touch panel 430 may include one or more touch sensors for detecting an input operation on an operation surface of the touch panel display screen. The touch panel 430 also detects a touch shape and a touch area. Used herein, the phrase “touch operation” refers to an input operation performed by touching an operation surface of the touch panel display with an instruction object, such as a finger, thumb, or stylus-type instrument. In the case where a stylus or the like is used in a touch operation, the stylus may include a conductive material at least at the tip of the stylus such that the sensors included in the touch panel 430 may detect when the stylus approaches/contacts the operation surface of the touch panel display (similar to the case in which a finger is used for the touch operation).

In certain aspects of the present disclosure, the touch panel 430 may be disposed adjacent to the display 420 (e.g., laminated) or may be formed integrally with the display 420. For simplicity, the present disclosure assumes the touch panel 430 is formed integrally with the display 420 and therefore, examples discussed herein may describe touch operations being performed on the surface of the display 420 rather than the touch panel 430. However, the skilled artisan will appreciate that this is not limiting.

For simplicity, the present disclosure assumes the touch panel 430 is a capacitance-type touch panel technology. However, it should be appreciated that aspects of the present disclosure may easily be applied to other touch panel types (e.g., resistance-type touch panels) with alternate structures. In certain aspects of the present disclosure, the touch panel 430 may include transparent electrode touch sensors arranged in the X-Y direction on the surface of transparent sensor glass.

The touch panel driver may be included in the touch panel 430 for control processing related to the touch panel 430, such as scanning control. For example, the touch panel driver may scan each sensor in an electrostatic capacitance transparent electrode pattern in the X-direction and Y-direction and detect the electrostatic capacitance value of each sensor to determine when a touch operation is performed. The touch panel driver may output a coordinate and corresponding electrostatic capacitance value for each sensor. The touch panel driver may also output a sensor identifier that may be mapped to a coordinate on the touch panel display screen. Additionally, the touch panel driver and touch panel sensors may detect when an instruction object, such as a finger is within a predetermined distance from an operation surface of the touch panel display screen. That is, the instruction object does not necessarily need to directly contact the operation surface of the touch panel display screen for touch sensors to detect the instruction object and perform processing described herein. For example, in an embodiment, the touch panel 430 may detect a position of a user's finger around an edge of the display panel 420 (e.g., gripping a protective case that surrounds the display/touch panel). Signals may be transmitted by the touch panel driver, e.g. in response to a detection of a touch operation, in response to a query from another element based on timed data exchange, etc.

The touch panel 430 and the display 420 may be surrounded by a protective casing, which may also enclose the other elements included in the user device 20. In an embodiment, a position of the user's fingers on the protective casing (but not directly on the surface of the display 420) may be detected by the touch panel 430 sensors. Accordingly, the controller 410 may perform display control processing described herein based on the detected position of the user's fingers gripping the casing. For example, an element in an interface may be moved to a new location within the interface (e.g., closer to one or more of the fingers) based on the detected finger position.

Further, in an embodiment, the controller 410 may be configured to detect which hand is holding the user device 20, based on the detected finger position. For example, the touch panel 430 sensors may detect a plurality of fingers on the left side of the user device 20 (e.g., on an edge of the display 420 or on the protective casing) and detect a single finger on the right side of the user device 20. In this exemplary scenario, the controller 410 may determine that the user is holding the user device 20 with his/her right hand because the detected grip pattern corresponds to an expected pattern when the user device 20 is held only with the right hand.

The operation key 440 may include one or more buttons or similar external control elements, which may generate an operation signal based on a detected input by the user. In addition to outputs from the touch panel 430, these operation signals may be supplied to the controller 410 for performing related processing and control. In certain aspects of the present disclosure, the processing and/or functions associated with external buttons and the like may be performed by the controller 410 in response to an input operation on the touch panel 430 display screen rather than the external button, key, etc. In this way, external buttons on the user device 20 may be eliminated in lieu of performing inputs via touch operations, thereby improving watertightness.

The antenna 406 may transmit/receive electromagnetic wave signals to/from other external apparatuses, and the short-distance wireless communication processor 407 may control the wireless communication performed between the other external apparatuses. Bluetooth, IEEE 802.11, and near-field communication (NFC) are non-limiting examples of wireless communication protocols that may be used for inter-device communication via the short-distance wireless communication processor 407.

The user device 20 may include a motion sensor 408. The motion sensor 408 may detect features of motion (i.e., one or more movements) of the user device 20. For example, the motion sensor 408 may include an accelerometer to detect acceleration, a gyroscope to detect angular velocity, a geomagnetic sensor to detect direction, a geo-location sensor to detect location, etc., or a combination thereof to detect motion of the user device 20. In an embodiment, the motion sensor 408 may generate a detection signal that includes data representing the detected motion. For example, the motion sensor 408 may determine a number of distinct movements in a motion (e.g., from start of the series of movements to the stop, within a predetermined time interval, etc.), a number of physical shocks on the user device 20 (e.g., a jarring, hitting, etc., of the electronic device), a speed and/or acceleration of the motion (instantaneous and/or temporal), or other motion features. The detected motion features may be included in the generated detection signal. The detection signal may be transmitted, e.g., to the controller 410, whereby further processing may be performed based on data included in the detection signal. The motion sensor 408 can work in conjunction with a Global Positioning System (GPS) section 460. The information of the present position detected by the GPS section 460 is transmitted to the controller 410. An antenna 461 is connected to the GPS section 460 for receiving and transmitting signals to and from a GPS satellite.

The user device 20 may include a camera section 409, which includes a lens and shutter for capturing photographs of the surroundings around the user device 20. In an embodiment, the camera section 409 captures surroundings of an opposite side of the user device 20 from the user. The images of the captured photographs can be displayed on the display panel 420. A memory section saves the captured photographs. The memory section may reside within the camera section 409 or it may be part of the memory 450. The camera section 409 can be a separate feature attached to the user device 20 or it can be a built-in camera feature.

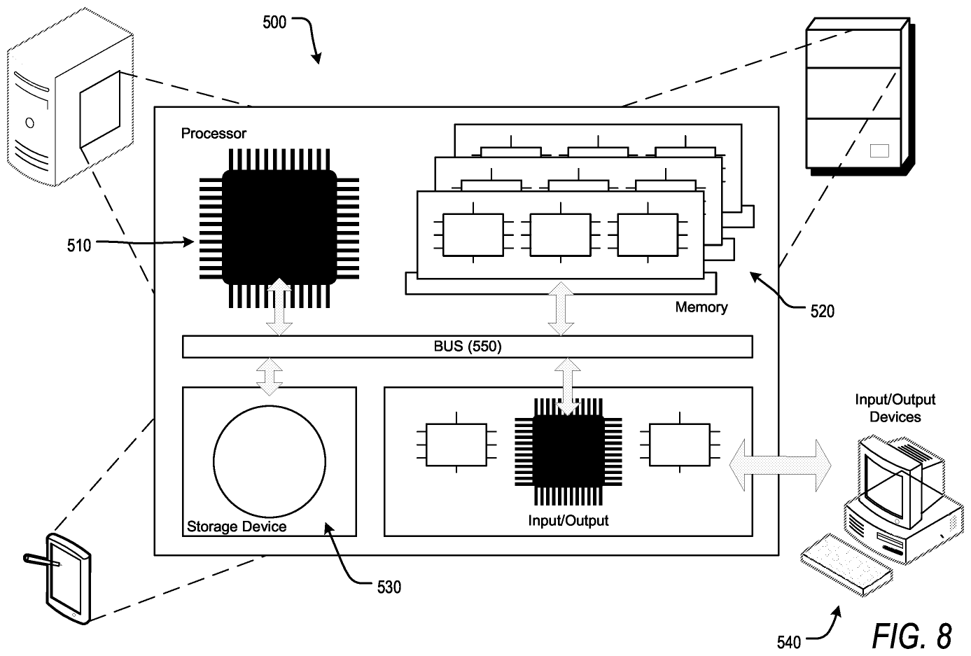

An example of a type of computer is shown in FIG. 8. The computer 500 can be used for the operations described in association with any of the computer-implement methods described previously, according to one implementation. The processing circuitry includes one or more of the elements discussed next with reference to FIG. 8. In FIG. 8, the computer 500 includes a processor 510, a memory 520, a storage device 530, and an input/output device 540. Each of the components 510, 520, 530, and 540 are interconnected using a system bus 550. The processor 510 is capable of processing instructions for execution within the system 500. In one implementation, the processor 510 is a single-threaded processor. In another implementation, the processor 510 is a multi-threaded processor. The processor 510 is capable of processing instructions stored in the memory 520 or on the storage device 530 to display graphical information for a user interface on the input/output device 540.

The memory 520 stores information within the computer 500. In one implementation, the memory 520 is a computer-readable medium. In one implementation, the memory 520 is a volatile memory unit. In another implementation, the memory 520 is a non-volatile memory unit.

The storage device 530 is capable of providing mass storage for the computer 500. In one implementation, the storage device 530 is a computer-readable medium. In various different implementations, the storage device 530 may be a floppy disk device, a hard disk device, an optical disk device, or a tape device.

The input/output device 540 provides input/output operations for the computer 500. In one implementation, the input/output device 540 includes a keyboard and/or pointing device. In another implementation, the input/output device 540 includes a display unit for displaying graphical user interfaces.

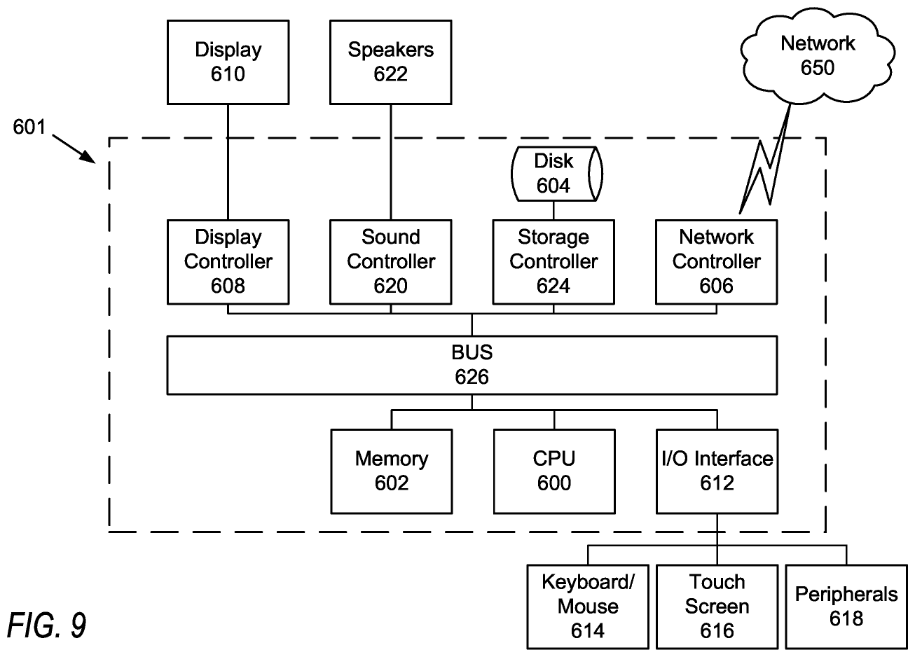

Next, a hardware description of a device 601 according to exemplary embodiments is described with reference to FIG. 9. In FIG. 9, the device 601 includes processing circuitry, as discussed above. The processing circuitry includes one or more of the elements discussed next with reference to FIG. 9. The process data and instructions may be stored in memory 602. These processes and instructions may also be stored on a storage medium disk 604 such as a hard drive (HDD) or portable storage medium or may be stored remotely. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the device 601 communicates, such as a server or computer.

Further, the claimed advancements may be provided as a utility application, background daemon, or component of an operating system, or combination thereof, executing in conjunction with CPU 600 and an operating system such as Microsoft Windows, UNIX, Solaris, LINUX, Apple MAC-OS and other systems known to those skilled in the art.

The hardware elements in order to achieve the device 601 may be realized by various circuitry elements, known to those skilled in the art. For example, CPU 600 may be a Xenon or Core processor from Intel of America or an Opteron processor from AMD of America, or may be other processor types that would be recognized by one of ordinary skill in the art. Alternatively, the CPU 600 may be implemented on an FPGA, ASIC, PLD or using discrete logic circuits, as one of ordinary skill in the art would recognize. Further, CPU 600 may be implemented as multiple processors cooperatively working in parallel to perform the instructions of the processes described above.

The device 601 in FIG. 9 also includes a network controller 606, such as an Intel Ethernet PRO network interface card from Intel Corporation of America, for interfacing with network 650, and to communicate with other devices. As can be appreciated, the network 650 can be a public network, such as the Internet, or a private network such as an LAN or WAN network, or any combination thereof and can also include PSTN or ISDN sub-networks. The network 650 can also be wired, such as an Ethernet network, or can be wireless such as a cellular network including EDGE, 3G, 4G and 5G wireless cellular systems. The wireless network can also be WiFi, Bluetooth, or any other wireless form of communication that is known.

The device 601 further includes a display controller 608, such as a NVIDIA Geforce GTX or Quadro graphics adaptor from NVIDIA Corporation of America for interfacing with display 610, such as an LCD monitor. A general purpose I/O interface 612 interfaces with a keyboard and/or mouse 614 as well as a touch screen panel 616 on or separate from display 610. General purpose I/O interface also connects to a variety of peripherals 618 including printers and scanners.

A sound controller 620 is also provided in the device 601 to interface with speakers/microphone 622 thereby providing sounds and/or music.

The general purpose storage controller 624 connects the storage medium disk 604 with communication bus 626, which may be an ISA, EISA, VESA, PCI, or similar, for interconnecting all of the components of the device 601. A description of the general features and functionality of the display 610, keyboard and/or mouse 614, as well as the display controller 608, storage controller 624, network controller 606, sound controller 620, and general purpose I/O interface 612 is omitted herein for brevity as these features are known.

While this specification contains many specific implementation details, these should not be construed as limitations on the scope of what may be claimed, but rather as descriptions of features that may be specific to particular embodiments.

Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

Embodiments of the present disclosure may also be set forth in the following parentheticals.

(1) A method for extracting a fetal electrocardiogram (ECG), comprising determining, via processing circuitry, a coherence between the maternal ECG and the abdominal ECG; attenuating, via the processing circuitry, the maternal ECG independent of the coherence; and extracting, via the processing circuitry, the fetal ECG from the abdominal ECG based on the coherence and the attenuated maternal ECG.

(2) The method of (1), further comprising dividing the maternal ECG and the abdominal ECG into epochs and extracting the fetal ECG for each of the epochs.

(3) The method of any (1) to (2), comprising determining the coherence between the maternal ECG and the abdominal ECG in the frequency domain.

(4) The method of any (1) to (3), wherein the maternal ECG is determined from the abdominal ECG using machine learning.

(5) The method of any (1) to (4), wherein the maternal ECG is attenuated based on a periodogram of the maternal ECG and a periodogram of the abdominal ECG.

(6) The method of any (1) to (5), further comprising calculating a loss function for the fetal ECG based on a number of missed heartbeats and a number of false heartbeats.

(7) The method of any (1) to (6), further comprising determining a signal quality of the fetal ECG using machine learning.

(8) The method of any (1) to (7), further comprising determining at least one fetal cardiac time interval based on the fetal ECG using machine learning.

(9) A device comprising processing circuitry configured to determine a coherence between a maternal ECG and an abdominal ECG, attenuate the maternal ECG independent of the coherence, and extract a fetal ECG from the abdominal ECG based on the coherence and the attenuated maternal ECG.

(10) The device of (9), wherein the processing circuitry is further configured to divide the maternal ECG and the abdominal ECG into epochs and extract the fetal ECG for each of the epochs.

(11) The device of any (9) to (10), wherein the processing circuitry is configured to determine the coherence between the maternal ECG and the abdominal ECG in the frequency domain.

(12) The device of any (9) to (11), wherein the maternal ECG is attenuated based on a periodogram of the maternal ECG and a periodogram of the abdominal ECG.

(13) The device of any (9) to (12), wherein the processing circuitry is further configured to calculate a loss function for the fetal ECG based on a number of missed heartbeats and a number of false heartbeats.

(14) The device of any (9) to (13), wherein the processing circuitry is further configured to determine at least one fetal cardiac interval based on the fetal ECG using machine learning.

(15) A non-transitory computer-readable storage medium for storing computer-readable instructions that, when executed by a computer, cause the computer to perform a method, the method comprising determining a coherence between a maternal ECG and an abdominal ECG, attenuating the maternal ECG independent of the coherence, and extracting the fetal ECG from the abdominal ECG based on the coherence and the attenuated maternal ECG.

(16) The non-transitory computer-readable storage medium of (15), further comprising dividing the maternal ECG and the abdominal ECG into epochs and extracting the fetal ECG for each of the epochs.

(17) The non-transitory computer-readable storage medium of any (15) to (16), comprising determining the coherence between the maternal ECG and the abdominal ECG in the frequency domain.

(18) The non-transitory computer-readable storage medium of any (15) to (17), wherein the maternal ECG is attenuated based on a periodogram of the maternal ECG and a periodogram of the abdominal ECG.

(19) The non-transitory computer-readable storage medium of any (15) to (18), further comprising calculating a loss function for the fetal ECG based on a number of missed heartbeats and a number of false heartbeats.

(20) The non-transitory computer-readable storage medium of any (15) to (19), further comprising determining at least one fetal cardiac time interval based on the fetal ECG using machine learning.

Thus, the foregoing discussion discloses and describes merely exemplary embodiments of the present disclosure. As will be understood by those skilled in the art, the present disclosure may be embodied in other specific forms without departing from the spirit thereof. Accordingly, the disclosure of the present disclosure is intended to be illustrative, but not limiting of the scope of the disclosure, as well as other claims. The disclosure, including any readily discernible variants of the teachings herein, defines, in part, the scope of the foregoing claim terminology such that no inventive subject matter is dedicated to the public.

1. A method for extracting a fetal electrocardiogram (ECG), comprising:
determining, via processing circuitry, a coherence between a maternal ECG and an abdominal ECG;
attenuating, via the processing circuitry, the maternal ECG independent of the coherence; and
extracting, via the processing circuitry, the fetal ECG from the abdominal ECG based on the coherence and the attenuated maternal ECG.

2. The method of claim 1, further comprising dividing the maternal ECG and the abdominal ECG into epochs and extracting the fetal ECG for each of the epochs.

3. The method of claim 1, comprising determining the coherence between the maternal ECG and the abdominal ECG in the frequency domain.

4. The method of claim 1, wherein the maternal ECG is determined from the abdominal ECG using machine learning.

5. The method of claim 1, wherein the maternal ECG is attenuated based on a periodogram of the maternal ECG and a periodogram of the abdominal ECG.

6. The method of claim 1, further comprising calculating a loss function for the fetal ECG based on a number of missed heartbeats and a number of false heartbeats.

7. The method of claim 1, further comprising determining a signal quality of the fetal ECG using machine learning.

8. The method of claim 1, further comprising determining at least one fetal cardiac time interval based on the fetal ECG using machine learning techniques.

9. A device comprising:
processing circuitry configured to:
determine a coherence between a maternal ECG and an abdominal ECG,
attenuate the maternal ECG independent of the coherence, and
extract a fetal ECG from the abdominal ECG based on the coherence and the attenuated maternal ECG.

10. The device of claim 9, wherein the processing circuitry is further configured to divide the maternal ECG and the abdominal ECG into epochs and extract the fetal ECG for each of the epochs.

11. The device of claim 9, wherein the processing circuitry is configured to determine the coherence between the maternal ECG and the abdominal ECG in the frequency domain.

12. The device of claim 9, wherein the maternal ECG is attenuated based on a periodogram of the maternal ECG and a periodogram of the abdominal ECG.

13. The device of claim 9, wherein the processing circuitry is further configured to calculate a loss function for the fetal ECG based on a number of missed heartbeats and a number of false heartbeats.

14. The device of claim 9, wherein the processing circuitry is further configured to determine at least one fetal cardiac time interval based on the fetal ECG using machine learning.

15. A non-transitory computer-readable storage medium for storing computer-readable instructions that, when executed by a computer, cause the computer to perform a method, the method comprising:
determining a coherence between the maternal ECG and the abdominal ECG;
attenuating the maternal ECG independent of the coherence; and
extracting fetal ECG from the abdominal ECG based on the coherence and the attenuated maternal ECG.

16. The non-transitory computer-readable storage medium of claim 15, further comprising dividing the maternal ECG and the abdominal ECG into epochs and extracting the fetal ECG for each of the epochs.

17. The non-transitory computer-readable storage medium of claim 15, comprising determining the coherence between the maternal ECG and the abdominal ECG in the frequency domain.

18. The non-transitory computer-readable storage medium of claim 15, wherein the maternal ECG is attenuated based on a periodogram of the maternal ECG and a periodogram of the abdominal ECG.

19. The non-transitory computer-readable storage medium of claim 15, further comprising calculating a loss function for the fetal ECG based on a number of missed heartbeats and a number of false heartbeats.

20. The non-transitory computer-readable storage medium of claim 15, further comprising determining at least one fetal cardiac time interval based on the fetal ECG using machine learning.
